(19) United States
(12) Patent Application Publication
(10) Pub. No.: US 2026/0345118 A1
(43) Pub. Date: Nov. 5, 2026

(54) INTRALUMINAL IMAGING DELIVERY ASSIST PLATFORM

(71) Applicant: CORTICAL SOLUTIONS, LLC, AUSTIN, TX (US)

(72) Inventor: Imran Farooqui, Austin, TX (US)

(21) Appl. No.: 19/668,258

(22) Filed: May 5, 2026

Related U.S. Application Data
(63) Continuation of application No. 19/667,575, filed on May 4, 2026.
(60) Provisional application No. 63/799,882, filed on May 5, 2025.

(51) Int. Cl. A61B 8/00; A61B 8/08; A61B 8/12; A61M 25/00; A61M 25/01

(52) U.S. Cl. CPC A61B 8/445; A61B 8/0808; A61B 8/0891; A61B 8/12; A61B 8/4209; A61B 8/44; A61M 25/0028; A61M 25/0169

(57) ABSTRACT

The present invention provides an intraluminal imaging delivery assist platform for neurovascular use. The platform may include a catheter shaft having an imaging lumen and a working lumen arranged side-by-side, a proximal hub assembly having a working port and an imaging port, an intravascular ultrasound imaging assembly disposed at least partially within the imaging lumen, and a distal tip region configured for atraumatic advancement through neurovascular anatomy. The working lumen may extend through an imaging region and the distal tip region to receive a microwire during over-the-wire advancement. After positioning the imaging region at a target neurovascular region, the microwire may be withdrawn and an acoustic coupling medium may be introduced into the working lumen to support acquisition of a substantially circumferential intraluminal ultrasound imaging data. The platform may support vessel assessment, lesion characterization, device apposition evaluation, guide catheter positioning, and neurovascular procedural decision-making.

### CLAIM OF PRIORITY

The present application is a Continuation patent application of and claims priority to a previously filed U.S. Non-Provisional patent application, namely, that having Serial No. 19/667,575 and a filing date of May. 4, 2026, and further, claims priority to a previously filed, U.S. Provisional patent application, namely, that having Serial No. 63/799,882 filed on May. 5, 2025, both of which are incorporated by reference herein in their entireties.

### BACKGROUND OF THE INVENTION

### Field of the Invention

The present invention relates generally to intraluminal imaging systems, catheter systems, and neurovascular access devices. More specifically, the present invention relates to catheter-based intraluminal imaging platforms configured for use within intracranial and extracranial neurovascular anatomy. The present invention further relates to delivery-assist catheter platforms incorporating intraluminal imaging functionality, including optical coherence tomography (OCT) imaging, intravascular ultrasound (IVUS) imaging, or other imaging modalities suitable for evaluating vessel morphology, vessel pathology, device apposition, and neurovascular treatment sites.

### Description of the Related Art

Neurovascular intervention frequently requires accurate assessment of vascular anatomy, lesion morphology, vessel wall condition, luminal narrowing, thrombotic material, aneurysmal structures, implanted devices, and adjacent anatomical constraints. The intracranial and extracranial vessels of the head and neck present unique procedural challenges due to their relatively small vessel diameters, tortuous pathways, fragile vessel walls, variable branching anatomy, and proximity to critical neurological structures. Procedures involving the internal carotid artery, middle cerebral artery, vertebral artery, basilar artery, and related vascular territories often require precise navigation through curved and narrowing access pathways. In these procedures, imaging information is typically used not only to diagnose or characterize pathology, but also to plan intervention, select device sizes, position guide catheters, evaluate treatment outcomes, and determine whether additional treatment steps are warranted. These requirements are particularly significant in connection with ischemic stroke associated with large or medium vessel occlusion, cerebral aneurysm evaluation and treatment planning, intracranial atherosclerotic disease, vasospasm, vasculitis, dissections, and other neurovascular conditions. As endovascular treatment techniques continue to advance, the need for more detailed intraluminal information within neurovascular anatomy has become increasingly important.

Conventional neurovascular imaging approaches often rely on imaging techniques performed outside the vessel lumen. Digital subtraction angiography, fluoroscopy, computed tomography angiography, magnetic resonance angiography, and related imaging modalities provide useful information regarding vessel geometry, contrast flow, gross luminal boundaries, and device position. These techniques, however, may provide limited information regarding the vessel wall itself, particularly where clinically relevant pathology exists within or adjacent to the wall rather than only within the contrast-filled lumen. Luminal imaging may underestimate plaque burden, wall remodeling, dissection planes, aneurysm neck morphology, thrombus organization, or vessel wall injury because these conditions can be difficult to visualize from a projection-based or external imaging perspective. In some circumstances, angiographic appearance may not fully reveal the underlying structure that determines treatment selection or device performance. As a result, neurovascular operators may need to infer important anatomical and pathological information from indirect imaging signs, procedural behavior, and pre-procedural imaging studies. This can increase uncertainty when selecting device size, evaluating device apposition, assessing residual stenosis, or determining whether a treated segment requires additional intervention.

Intravascular imaging technologies have been developed and used extensively in other vascular fields, particularly in coronary and peripheral vascular applications. Optical coherence tomography can provide high-resolution optical imaging of superficial vessel structures, including fine luminal and near-wall features. Intravascular ultrasound can provide deeper acoustic imaging of vessel wall architecture, plaque burden, and vessel remodeling. These modalities provide complementary forms of information and have significantly influenced diagnostic and procedural decision-making in vascular beds where compatible imaging catheter systems are available. However, technologies designed for coronary or peripheral use are not automatically suitable for neurovascular use. Neurovascular anatomy requires different dimensional constraints, flexibility characteristics, shaft behavior, distal tip configurations, and access compatibility. A catheter that functions adequately in the coronary circulation may be too large, too stiff, insufficiently navigable, or insufficiently compatible with intracranial access pathways.

The translation of intraluminal imaging to the neurovascular field presents a number of technical barriers. Neurovascular vessels may include sharp curves, branching geometries, and small target segments that limit the ability to advance imaging devices safely. The need for distal access catheter compatibility imposes strict limits on outer diameter, wall thickness, lumen arrangement, and internal component routing. In addition, neurovascular procedures frequently require a delivery-assist function, meaning that an access or support catheter must help maintain stable access, support device advancement, or position a guide catheter or intermediate catheter in a manner that enables a subsequent intervention. Conventional intraluminal imaging catheters generally focus on imaging alone and may not provide the pushability, trackability, torque response, tip behavior, and guide-catheter support needed for neurovascular delivery assistance. Conversely, conventional delivery-assist catheters may be configured for navigation and support, but do not generally provide integrated intraluminal imaging capability. This separation of functions can require multiple devices, device exchanges, or procedural compromises.

Device exchange can be especially undesirable in neurovascular procedures. When a catheter is exchanged, the operator may risk losing access, changing device position, disturbing thrombotic or fragile tissue, extending procedure time, or increasing the number of manipulations within delicate vessels. Even when exchanges are technically successful, the cumulative procedural burden can increase complexity and reduce efficiency. In an ischemic stroke procedure, time delays may be clinically meaningful because treatment windows can be narrow and neurological outcomes may depend on rapid restoration of flow. In aneurysm procedures, device placement may require careful assessment of neck geometry, parent vessel relationship, branch incorporation, and device apposition. In intracranial atherosclerosis, evaluation of stenosis severity and vessel wall condition may influence whether angioplasty, stenting, medication, or another management pathway is appropriate. In vasospasm, dissection, or vasculitis, intraluminal and vessel-wall information may assist with distinguishing mechanical narrowing, inflammatory change, vessel injury, thrombus, and treatment response. A platform that reduces unnecessary exchanges while providing useful intraluminal information would therefore address a meaningful procedural need.

Another challenge arises from the different physical requirements of OCT and IVUS. OCT relies on near-infrared light and generally requires a relatively clear optical path to obtain high-quality images. In a catheter system, the placement of adjacent lumens, wall material, reinforcing structures, or metallic components can obstruct or degrade optical transmission. A working lumen positioned adjacent to an OCT imaging path may create a shadow, dead zone, or incomplete field of view if optical energy cannot pass through the intervening material with sufficient fidelity. For neurovascular use, incomplete circumferential imaging may be problematic because pathology may be eccentric or asymmetric around the vessel circumference. Plaque, dissection, thrombus, device malapposition, and aneurysm-related features may be localized to one side of the vessel rather than uniformly distributed. Accordingly, an OCT-based neurovascular imaging catheter may require architecture that preserves a substantially unobstructed circumferential optical imaging field while still permitting safe wire-guided navigation.

IVUS presents a different set of design considerations. Unlike OCT, IVUS uses acoustic energy rather than optical energy, and acoustic waves may tolerate certain adjacent-lumen structures that would interfere with optical imaging. Acoustic coupling media, including saline or other suitable fluids, may improve transmission between transducers, catheter materials, and surrounding blood or tissue. However, an IVUS catheter still must accommodate a drive cable, transducer assembly, working lumen, guidewire, hub connections, and sufficient wall structure within a very small overall profile. The catheter must also maintain flexibility and support across its length while preserving a distal configuration suitable for neurovascular navigation. Where a working lumen and imaging lumen are arranged side by side, the geometry of the lumens, intervening septum, outer jacket, reinforcement pattern, acoustic coupling pathway, and distal tip transition can influence imaging performance and navigational behavior. A design that merely combines a generic IVUS imaging lumen with a generic working lumen may not provide the necessary balance of acoustic imaging performance, guidewire compatibility, and delivery-assist functionality.

In addition to the distal imaging region, the proximal hub architecture can affect device usability and performance. A catheter that includes separate imaging and working paths must route those paths from the catheter body into corresponding proximal port structures. The hub must accommodate imaging data transmission, drive cable routing, flushing, contrast injection, acoustic coupling medium delivery, guidewire access, or other procedural functions depending on the imaging modality. If hub paths are poorly arranged, the device may become difficult to prepare, flush, connect, or operate. The transition from catheter shaft to strain relief to hub body must also preserve lumen alignment and avoid undesirable kinking, leakage, interference, or cross-communication between paths. In neurovascular intervention, where device preparation and procedural timing can be significant, a hub architecture that supports reliable operation without adding unnecessary ports or workflow complexity can be valuable.

Distal tip design is also particularly important in the neurovascular field. The distal tip must be atraumatic enough for use in delicate vessels while still providing sufficient support and directional behavior for advancement. A blunt, stiff, or poorly tapered distal structure may increase the risk of vessel injury or may fail to track through tortuous anatomy. A tip that is too soft or unsupported may buckle, prolapse, or fail to follow a microwire. In devices that include multiple lumens or transition from one lumen arrangement to another, the distal tip may need to reconcile internal geometry changes with external profile requirements. For example, a working lumen may need to continue through the distal tip while an imaging lumen terminates, or a wire channel may need to exit before an imaging zone to preserve imaging clearance. These transitions can create changes in material distribution, bending response, and local stiffness. The resulting geometry may affect how the distal region behaves during advancement, pullback, imaging, and withdrawal.

There is also a need to balance imaging-zone length, catheter outer diameter, lumen size, and anatomical coverage. A longer imaging zone may permit broader anatomical evaluation during a single pass, but may reduce flexibility or increase the length of a stiffer distal region. A shorter imaging zone may improve trackability but require additional pullback or repositioning. A larger outer diameter may provide more room for imaging components and guidewire lumens, but may reduce compatibility with distal access catheters and smaller neurovascular vessels. A smaller outer diameter may improve access but can impose manufacturing and performance limitations. In neurovascular anatomy, target vessels may include regions of the internal carotid artery, M1 or M2 segments of the middle cerebral artery, vertebrobasilar anatomy, or other vessels with differing diameters and curvature. These anatomical constraints require careful integration of imaging components, lumen geometry, material selection, reinforcement, coating, and tip design.

Radiopacity, markers, and positional feedback further contribute to procedural performance. In many neurovascular procedures, fluoroscopy remains a primary visualization tool for catheter advancement and positioning. Radiopaque markers can help identify the distal region, imaging zone, tip location, or pullback start point. Without reliable visual indicators, the operator may have difficulty correlating intraluminal imaging data with angiographic location or with a specific treatment site. Markers must be positioned in a manner that provides useful guidance without interfering with optical or acoustic imaging performance. Metallic elements may create artifacts, shadows, or stiffness changes if located improperly. Therefore, marker placement and material selection may require coordination with imaging architecture and catheter flexibility requirements.

Workflow considerations also influence the practical value of a neurovascular intraluminal imaging catheter. A system may need to support vascular access, guide catheter advancement, target site identification, platform preparation, guidewire loading, catheter advancement, imaging media delivery, imaging acquisition, pullback, and post-imaging decisions. Depending on the architecture, the microwire may remain in place during imaging, be withdrawn before imaging, or exit through a monorail transition. A working port may serve multiple functions at different procedural stages, such as guidewire access during navigation and fluid delivery during imaging. The imaging port may connect to an imaging console, drive system, processing module, or display. The ability to perform these steps in a consistent sequence may reduce setup complexity and improve adoption. If a catheter platform can integrate imaging with familiar neurovascular access steps, operators may be more likely to use intraluminal imaging during procedures where it can improve diagnostic confidence and planning.

The need for a neurovascular-specific intraluminal imaging delivery-assist platform is further reinforced by the evolving role of endovascular therapy. Device-based treatments for stroke, aneurysm, stenosis, vasospasm, and other vascular conditions increasingly require precise deployment and assessment. Flow diverters, stents, thrombectomy devices, angioplasty balloons, intrasaccular devices, and other systems may benefit from more accurate knowledge of vessel size, lesion morphology, vessel wall condition, and device-vessel interaction. Post-treatment imaging may help assess apposition, residual narrowing, thrombus, dissection, vessel remodeling, aneurysm neck residual, or other features that influence immediate or follow-up management. While external imaging techniques remain important, intraluminal imaging may provide a more direct evaluation of structures that are otherwise difficult to characterize. A catheter platform capable of supporting both imaging and delivery-assist functions can therefore provide value across diagnosis, planning, peri-procedural assessment, and longitudinal management.

Potential solutions may involve intraluminal imaging catheter platforms specifically dimensioned and configured for neurovascular anatomy rather than adapted from larger vascular systems. Such platforms may integrate an imaging modality into a delivery-assist catheter architecture while preserving guidewire compatibility, access catheter compatibility, atraumatic navigation, and reliable proximal connection interfaces. Different imaging modalities may require different catheter architectures. For example, an optical imaging implementation may benefit from a geometry that removes an adjacent working lumen from the imaging zone to preserve circumferential optical access, while an ultrasound implementation may permit a dual-lumen structure if acoustic coupling and signal processing can address adjacent-lumen effects. A flexible patent specification should therefore account for multiple imaging architectures while maintaining a common platform concept directed to intraluminal neurovascular imaging and delivery assistance. There remains a need for catheter systems that combine these features in a clinically usable form suitable for neurovascular access, intraluminal imaging, guide-catheter support, and procedural workflow integration.

### SUMMARY OF THE INVENTION

The present invention provides a neurovascular intraluminal imaging delivery assist platform configured to integrate intraluminal vessel imaging with catheter-based delivery assistance in a single platform suitable for intracranial and extracranial neurovascular anatomy. In one implementation, the platform may comprise an optical coherence tomography (OCT) delivery assist catheter having a catheter body shaft, a monorail transition zone, an imaging zone, and a distal tip zone, wherein the catheter body shaft may include a vertically arranged dual-lumen or dual-extrusion region having an imaging lumen and a wire channel arranged in a dorsal-ventral relationship. The wire channel may terminate at or near the monorail transition zone such that a microwire exits the catheter before the imaging zone, thereby allowing the imaging zone to house an OCT imaging assembly with reduced optical obstruction and substantially circumferential imaging capability. In another implementation, the platform may comprise an intravascular ultrasound (IVUS) delivery assist catheter having an over-the-wire dual-lumen architecture in which an imaging lumen and a working lumen are arranged in a side-by-side oval-core configuration within a continuous outer polymer jacket. The IVUS implementation may maintain the microwire within the working lumen through the distal tip region during navigation, while the imaging lumen houses an IVUS drive cable and imaging assembly configured for rotational acoustic imaging. Across the various implementations, the platform may include a proximal hub assembly having working and imaging ports, one or more strain relief regions, one or more flushing or coupling-fluid pathways, one or more radiopaque markers, a flexible atraumatic distal tip, and a shaft construction dimensioned and configured for compatibility with neurovascular guide catheters, distal access catheters, and tortuous neurovascular anatomy.

The present invention provides several advantages over conventional imaging catheters, guide catheters, and delivery-assist systems. Conventional neurovascular procedures often rely on external imaging modalities that provide useful luminal and positional information but may not adequately characterize vessel wall morphology, lesion composition, device apposition, thrombus, dissection, residual stenosis, aneurysm neck configuration, or other intraluminal features that may affect procedural planning and treatment decisions. In addition, conventional intraluminal imaging catheters are generally developed for coronary or peripheral vascular environments and may lack the profile, flexibility, navigational behavior, distal tip design, and guide-catheter compatibility required for intracranial and extracranial neurovascular use. Conventional delivery-assist catheters may provide navigational support but generally do not provide integrated intraluminal imaging, thereby requiring separate imaging and delivery devices or multiple device exchanges in procedures where intraluminal assessment is desired. By integrating imaging functionality into a delivery-assist platform, the present invention may reduce procedural complexity, support more accurate lesion and vessel assessment, facilitate guide catheter positioning, and enable image-informed decision-making before, during, or after a neurovascular intervention. The platform may further improve procedural efficiency by allowing a single catheter architecture to support both navigation and intraluminal assessment, while preserving compatibility with microwires and access systems commonly used in neurovascular procedures.

In certain OCT implementations, the present invention provides an additional benefit by addressing the optical limitations that can arise when a working lumen is positioned adjacent to an optical imaging path. Because OCT relies on light transmission, adjacent catheter structures may create optical shadowing, dead zones, or incomplete imaging coverage if they remain within the imaging field. The monorail transition configuration of the OCT implementation may permit the microwire to exit the catheter proximal to the imaging zone, allowing the imaging zone to be configured as a single-lumen or substantially unobstructed imaging region. This arrangement may enable substantially circumferential OCT imaging while maintaining wire-guided navigation through the catheter body shaft and delivery-assist behavior during advancement. As a result, the OCT implementation may provide high-resolution intraluminal imaging while still maintaining the navigational and support features expected from a neurovascular delivery-assist catheter. This combination may be particularly beneficial in evaluating asymmetric neurovascular pathology, including eccentric plaque, dissection planes, thrombus, device malapposition, aneurysm neck features, and other localized vessel wall conditions.

In certain IVUS implementations, the present invention provides a different but complementary benefit by using the acoustic properties of ultrasound to maintain a full-length over-the-wire dual-lumen arrangement while still enabling intraluminal imaging. The IVUS implementation may include an oval-core dual-lumen configuration in which the imaging lumen and working lumen are arranged side-by-side within a continuous outer polymer jacket, thereby preserving guidewire access through the catheter while also routing an IVUS imaging assembly through the imaging lumen. Because IVUS uses acoustic energy rather than optical energy, the adjacent working lumen may be accommodated through suitable catheter geometry, acoustic coupling media, and signal-processing considerations. In some implementations, the working port may serve sequential functions, including microwire access during navigation and acoustic coupling medium delivery during imaging, thereby reducing the need for additional ports or separate procedural devices. The working lumen may also transition toward a centered position in the distal tip zone after termination of the imaging lumen, thereby supporting atraumatic distal navigation and improved distal tip behavior. This IVUS architecture may preserve the delivery-assist advantages of an over-the-wire catheter while adding intraluminal ultrasound imaging capability in a neurovascular-compatible form.

In a first implementation of the present invention, an intraluminal imaging delivery assist platform for neurovascular use may include a catheter shaft configured for advancement through a neurovascular guide catheter, the catheter shaft defining an imaging lumen and a wire channel, a proximal hub assembly coupled to the catheter shaft, the proximal hub assembly including a working port in communication with the wire channel and an imaging port in communication with the imaging lumen, a wire exit region disposed along the catheter shaft and configured to direct a microwire from the wire channel to an exterior of the catheter shaft, an optical coherence tomography imaging assembly disposed at least partially within the imaging lumen and configured to acquire circumferential intraluminal imaging data from an imaging region located distal to the wire exit region, and a distal tip region disposed distal to the imaging region and configured for atraumatic advancement through neurovascular anatomy. The wire channel may be configured to receive the microwire during advancement of the catheter shaft through intracranial or extracranial neurovascular anatomy. The catheter shaft may be configured to provide wire-guided delivery assistance while positioning the optical coherence tomography imaging assembly for intraluminal imaging within the neurovascular anatomy.

In another implementation of the present invention, an intraluminal imaging delivery assist platform for neurovascular use may include a catheter shaft configured for advancement through a neurovascular guide catheter, the catheter shaft including an imaging lumen and a working lumen arranged side-by-side within at least a portion of the catheter shaft, a proximal hub assembly coupled to the catheter shaft, the proximal hub assembly including a working port in communication with the working lumen and an imaging port in communication with the imaging lumen, an intravascular ultrasound imaging assembly disposed at least partially within the imaging lumen and configured to acquire circumferential intraluminal ultrasound imaging data from an imaging region of the catheter shaft, a distal tip region disposed distal to the imaging region and configured for atraumatic advancement through neurovascular anatomy, and a microwire receivable within the working lumen during advancement of the catheter shaft through intracranial or extracranial neurovascular anatomy. The working lumen may extend through the imaging region such that the catheter shaft is configured for over-the-wire advancement through the neurovascular guide catheter while the intravascular ultrasound imaging assembly is positioned for intraluminal ultrasound imaging. The proximal hub assembly may be configured to permit introduction of an acoustic coupling medium into at least one of the imaging lumen or the working lumen to support acquisition of the circumferential intraluminal ultrasound imaging data.

In another aspect, the wire exit region may be disposed proximal to the imaging region such that the microwire exits the wire channel before reaching the imaging region.

In another aspect, the wire exit region may include a monorail transition zone configured to transition the catheter shaft from an over-the-wire configuration proximal to the monorail transition zone to a monorail configuration distal to the monorail transition zone.

In another aspect, the wire exit region may include a ventral opening formed through a ventral portion of the catheter shaft.

In another aspect, the ventral opening may be defined at least partially by a contoured ventral relief configured to guide the microwire from the wire channel to the exterior of the catheter shaft.

In another aspect, the wire channel may terminate proximal to the imaging region.

In another aspect, the imaging region may be configured such that the wire channel does not extend through the imaging region.

In another aspect, the imaging region may include a single-lumen region housing the optical coherence tomography imaging assembly.

In another aspect, the imaging lumen and the wire channel may be arranged in a dorsal-ventral configuration within at least a portion of the catheter shaft proximal to the wire exit region.

In another aspect, the imaging lumen may be positioned dorsally relative to the wire channel and the wire channel may be positioned ventrally relative to the imaging lumen.

In another aspect, the catheter shaft may include a continuous outer polymer jacket extending along the catheter shaft through the wire exit region and the imaging region.

In another aspect, the continuous outer polymer jacket may include one or more zone-specific material properties selected from different durometers, different polymer compositions, different reinforcement densities, or combinations thereof.

In another aspect, the imaging region may have an outer diameter smaller than an outer diameter of a portion of the catheter shaft proximal to the wire exit region.

In another aspect, the optical coherence tomography imaging assembly may include an optical drive cable and an optical imaging component configured for rotational imaging within the imaging lumen.

In another aspect, the optical coherence tomography imaging assembly may be configured to acquire substantially circumferential imaging data around the imaging region.

In another aspect, the platform may include at least one radiopaque marker disposed adjacent to the imaging region or the distal tip region.

In another aspect, the distal tip region may include a tapered atraumatic distal tip configured to navigate intracranial neurovascular anatomy.

In another aspect, the distal tip region may include a vestigial lumen extending distally from the imaging region into at least a portion of the tapered atraumatic distal tip.

In another implementation of the present invention, an intraluminal imaging delivery assist catheter for neurovascular use may include a catheter body shaft configured for advancement through a neurovascular guide catheter, the catheter body shaft including an imaging lumen and a wire channel arranged in a dorsal-ventral relationship within at least a portion of the catheter body shaft, a proximal hub assembly coupled to the catheter body shaft, the proximal hub assembly including a working port in communication with the wire channel and an imaging port in communication with the imaging lumen, a monorail transition zone disposed distal to the catheter body shaft, the monorail transition zone including a ventral wire exit opening through which the wire channel communicates with an exterior of the intraluminal imaging delivery assist catheter, an imaging zone disposed distal to the monorail transition zone, the imaging zone including the imaging lumen and being configured such that the wire channel does not extend through the imaging zone, an optical coherence tomography imaging assembly disposed at least partially within the imaging lumen in the imaging zone and configured to acquire substantially circumferential intraluminal imaging data from within a neurovascular vessel, and a distal tip zone disposed distal to the imaging zone, the distal tip zone including a tapered atraumatic distal tip configured for advancement through tortuous neurovascular anatomy. The wire channel may be configured to receive a microwire during advancement of the intraluminal imaging delivery assist catheter. The monorail transition zone may be configured to direct the microwire from the wire channel to the exterior of the intraluminal imaging delivery assist catheter before the microwire reaches the imaging zone. The imaging zone may be configured to provide a substantially unobstructed optical imaging field for the optical coherence tomography imaging assembly after the microwire exits through the ventral wire exit opening.

In another implementation of the present invention, a method of performing intraluminal neurovascular imaging using an intraluminal imaging delivery assist catheter may include advancing a neurovascular guide catheter through vasculature of a patient toward a target neurovascular region, advancing an intraluminal imaging delivery assist catheter through the neurovascular guide catheter over a microwire, the intraluminal imaging delivery assist catheter including an imaging lumen, a wire channel, a wire exit region, an optical coherence tomography imaging assembly, an imaging region distal to the wire exit region, and a distal tip region, directing the microwire from the wire channel to an exterior of the intraluminal imaging delivery assist catheter at the wire exit region before the microwire reaches the imaging region, positioning the imaging region within or adjacent to the target neurovascular region, acquiring, using the optical coherence tomography imaging assembly, circumferential intraluminal imaging data from the target neurovascular region, using the circumferential intraluminal imaging data to assess at least one of vessel morphology, vessel pathology, stenosis, thrombus, dissection, aneurysm neck configuration, vessel diameter, or device apposition, and using the intraluminal imaging delivery assist catheter to provide wire-guided delivery assistance during positioning of the neurovascular guide catheter or during assessment of the target neurovascular region.

In another implementation of the present invention, a method of performing intraluminal neurovascular imaging using an intraluminal imaging delivery assist catheter may include advancing a neurovascular guide catheter through vasculature of a patient toward a target neurovascular region, advancing the intraluminal imaging delivery assist catheter through the neurovascular guide catheter over a microwire, the intraluminal imaging delivery assist catheter including an imaging lumen, a working lumen arranged side-by-side with the imaging lumen, an intravascular ultrasound imaging assembly disposed at least partially within the imaging lumen, and a distal tip region, positioning an imaging region of the intraluminal imaging delivery assist catheter within or adjacent to the target neurovascular region, withdrawing the microwire from the working lumen after positioning the imaging region within or adjacent to the target neurovascular region, introducing an acoustic coupling medium into the working lumen after withdrawing the microwire from the working lumen, acquiring, using the intravascular ultrasound imaging assembly, circumferential intraluminal ultrasound imaging data from the target neurovascular region, and using the circumferential intraluminal ultrasound imaging data to assess at least one of vessel morphology, vessel pathology, stenosis, thrombus, dissection, aneurysm neck configuration, vessel diameter, or device apposition.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is an anatomical system overview illustrating an intraluminal imaging delivery assist platform deployed through a neurovascular access pathway toward a target site according to one implementation of the present invention;

FIG. 2 is a longitudinal view illustrating the intraluminal imaging delivery assist platform of FIG. 1, including a proximal hub assembly, a catheter body shaft, a monorail transition zone, an imaging zone, and a distal tip region according to one implementation of the present invention;

FIG. 3 is an enlarged longitudinal view illustrating a distal critical zone of the intraluminal imaging delivery assist platform of FIG. 2, including the monorail transition zone, the imaging zone, the distal tip region, a microwire, and an optical coherence tomography imaging assembly according to one implementation of the present invention;

FIG. 4 is a series of cross-sectional views illustrating representative internal configurations of the intraluminal imaging delivery assist platform of FIG. 2 through the catheter body shaft, the monorail transition zone, the imaging zone, and the distal tip region according to one implementation of the present invention;

FIG. 5 is an enlarged longitudinal view illustrating the proximal hub assembly of the intraluminal imaging delivery assist platform of FIG. 2, including a hub body, a strain relief, a working port, an imaging port, and a webbing zone according to one implementation of the present invention;

FIG. 6 is a series of cross-sectional views illustrating representative internal configurations of the proximal hub assembly of FIG. 5, including lumen routing through the catheter body shaft, the strain relief, the hub body, the working port, and the imaging port according to one implementation of the present invention;

FIG. 7 is a workflow diagram illustrating a method of using the intraluminal imaging delivery assist platform to establish vascular access, advance the platform toward a target site, perform intraluminal imaging, and support neurovascular procedural decision-making according to one implementation of the present invention;

FIG. 8 is an anatomical system overview illustrating an intraluminal imaging delivery assist platform deployed through a neurovascular access pathway toward a target site according to another implementation of the present invention;

FIG. 9A is a longitudinal side view illustrating the intraluminal imaging delivery assist platform of FIG. 8, including a proximal hub assembly, a catheter body shaft, an imaging zone, a distal tip zone, and an intravascular ultrasound imaging assembly according to another implementation of the present invention;

FIG. 9B is a longitudinal top view illustrating the intraluminal imaging delivery assist platform of FIG. 9A, including a dual-lumen arrangement extending through the catheter body shaft and imaging zone according to another implementation of the present invention;

FIG. 10 is a series of cross-sectional views illustrating representative internal configurations of the intraluminal imaging delivery assist platform of FIGS. 9A-9B through the catheter body shaft, the imaging zone, and the distal tip zone according to another implementation of the present invention;

FIG. 11 is an enlarged longitudinal view illustrating a proximal hub assembly of the intraluminal imaging delivery assist platform of FIGS. 9A-9B, including a hub body, a strain relief, a working port, an imaging port, and a webbing zone according to another implementation of the present invention;

FIG. 12 is a series of cross-sectional views illustrating representative internal configurations of the proximal hub assembly of FIG. 11, including lumen routing through the catheter body shaft, the strain relief, the hub body, the working port, and the imaging port according to another implementation of the present invention; and

FIG. 13 is a workflow diagram illustrating a method of using the intraluminal imaging delivery assist platform of FIG. 8 to establish vascular access, advance the platform toward a target site, perform intravascular ultrasound imaging, and support neurovascular procedural decision-making according to another implementation of the present invention.

Like reference numerals refer to like parts throughout the several views of the drawings.

### DETAILED DESCRIPTION OF THE PREFERRED EMBODIMENT

The following detailed description is provided to describe illustrative implementations of the invention with reference to the accompanying drawings. The structures, dimensions, lumen arrangements, port configurations, imaging components, material selections, workflow steps, and anatomical use cases described herein are provided as non-limiting examples and may be modified, substituted, combined, omitted, rearranged, or supplemented without departing from the scope of the claimed invention. Directional terms such as proximal, distal, dorsal, ventral, upper, lower, side, top, bottom, lateral, longitudinal, axial, radial, circumferential, inward, and outward are used for convenience with reference to the orientations shown in the drawings and are not intended to require a fixed orientation during manufacturing, packaging, storage, preparation, insertion, navigation, imaging, or withdrawal. The term proximal may generally refer to a portion of a device nearer to an operator during use, and the term distal may generally refer to a portion farther from the operator and nearer to a target site within a patient. The term neurovascular anatomy may refer to intracranial vessels, extracranial vessels of the head and neck, carotid circulation, vertebrobasilar circulation, access pathways leading to intracranial circulation, or other vascular regions associated with neurovascular diagnosis or intervention. The term intraluminal imaging may refer to imaging performed from within a vessel lumen using an imaging component carried by, supported by, disposed in, or otherwise associated with a catheter platform. The term delivery assist may refer to a catheter configuration that may support advancement, navigation, positioning, access maintenance, guide catheter placement, distal access, imaging access, or procedural support within a vascular pathway, and does not require that the platform itself deploy a therapeutic device.

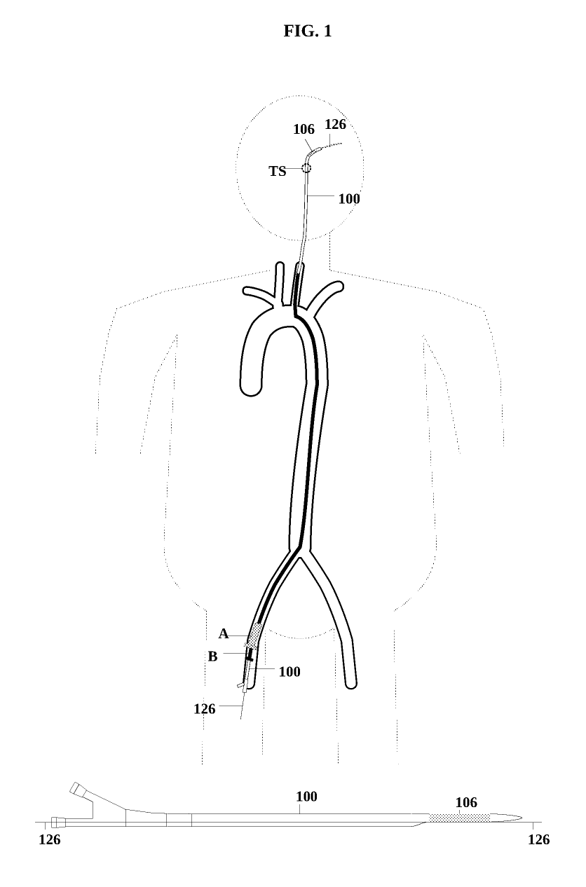

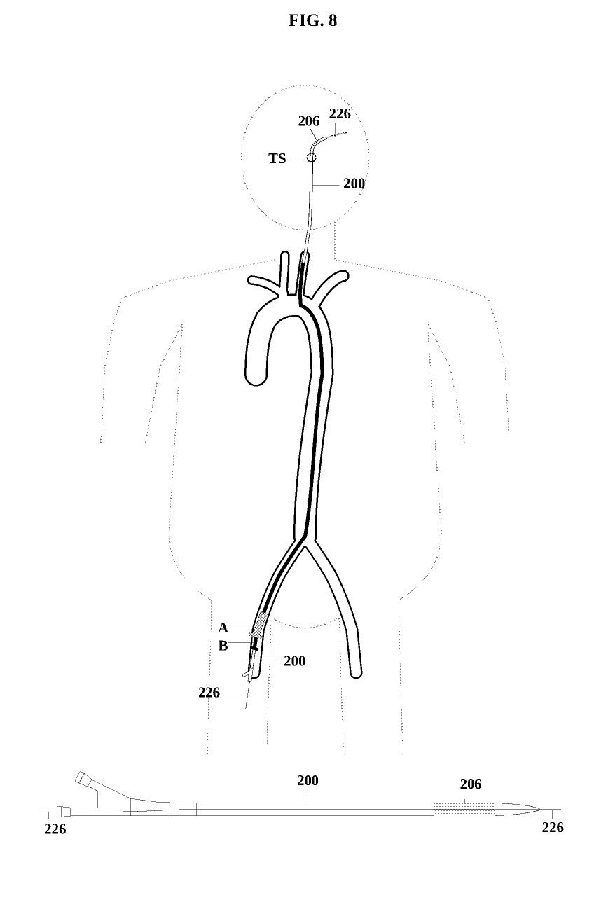

Referring generally to FIGS. 1-13, the present invention may provide an intraluminal imaging delivery assist platform configured for neurovascular use. The platform may integrate intraluminal imaging capability with catheter-based delivery assistance so that the platform can be advanced through tortuous neurovascular anatomy while also acquiring imaging data from within a vessel or target vascular region. A first implementation, shown generally in FIGS. 1-7 and identified with reference numerals beginning at 100, may be configured as an optical coherence tomography (OCT) delivery assist catheter having a hybrid over-the-wire-to-monorail architecture. A second implementation, shown generally in FIGS. 8-13 and identified with reference numerals beginning at 200, may be configured as an intravascular ultrasound (IVUS) delivery assist catheter having a full-length over-the-wire dual-lumen architecture. The first and second implementations may share a common platform concept directed to combining intraluminal imaging with neurovascular delivery-assist navigation, while also using different architecture choices based on modality-specific physics. OCT imaging may use near-infrared light and may benefit from removal of an adjacent working or wire lumen from the imaging field. IVUS imaging may use acoustic energy and may accommodate an adjacent working lumen when acoustic coupling media, lumen geometry, material selection, and signal processing are coordinated. Features described with respect to one implementation may be used with another implementation where technically compatible, unless the context indicates otherwise.

Referring now to FIG. 1, an anatomical system overview is shown for a first implementation of an intraluminal imaging delivery assist platform 100 deployed through a neurovascular access pathway toward a target site TS. An access sheath A may be positioned percutaneously through a femoral access location, and a guide catheter B may be advanced through access sheath A and through a vascular pathway toward a target great vessel. The vascular pathway may extend through the femoral artery, iliac artery, aorta, aortic arch, carotid artery, vertebral artery, or another vascular route suitable for accessing intracranial or extracranial neurovascular anatomy. The platform 100 may be advanced through guide catheter B toward target site TS. Target site TS may correspond to a location within one or more segments of the internal carotid artery, including the cervical, petrous, cavernous, paraclinoid, or supraclinoid segments, the middle cerebral artery, the anterior cerebral artery, the vertebral artery, the basilar artery, or another neurovascular vessel or branch. Target site TS may be associated with stenosis, thrombus, embolus, aneurysm, dissection, vasospasm, vasculitis, vessel sizing, implanted device apposition, post-treatment assessment, or another condition or procedural objective. An imaging zone 106 of platform 100 may be positioned at, near, across, or adjacent to target site TS so that intraluminal OCT imaging data may be acquired from within the vessel.

Still referring to FIG. 1, platform 100 may be advanced over or in cooperation with a microwire 126. Microwire 126 may provide a navigation path through tortuous vascular anatomy and may extend distally beyond at least a portion of platform 100 during advancement. Platform 100 may be dimensioned and configured to pass through guide catheter B while maintaining flexibility, pushability, trackability, and torque response suitable for neurovascular navigation. In some implementations, guide catheter B may comprise a neurovascular guide catheter, intermediate catheter, distal access catheter, aspiration catheter, or other access catheter suitable for placement in or near a target great vessel. Platform 100 may be configured for compatibility with distal access catheters having an internal diameter greater than approximately 0.068 inches to approximately 0.070 inches, and may be compatible with access systems in approximately the 0.070 inch to approximately 0.074 inch class. Platform 100 may have a working length of approximately 140 cm to approximately 170 cm, and in one proposed configuration may have a working length of approximately 155 cm. Other lengths may be used based on access route, target anatomy, hub configuration, or physician preference. Although FIG. 1 depicts a transfemoral approach, platform 100 may also be used with radial, brachial, direct carotid, or other clinically suitable access routes.

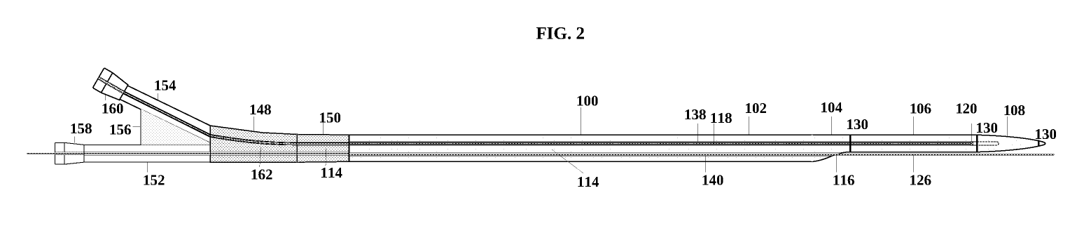

Referring now to FIG. 2, platform 100 may include a catheter body shaft 102, a monorail transition zone 104, imaging zone 106, and a distal tip zone 108 arranged generally from proximal to distal. Catheter body shaft 102 may be coupled to a proximal hub assembly and may define one or more internal paths for imaging and microwire navigation. Catheter body shaft 102 may have an outer diameter of approximately 0.060 inches to approximately 0.072 inches, with approximately 0.065 inches being one proposed value suitable for compatibility with certain neurovascular access platforms. Catheter body shaft 102 may define an imaging lumen 110 and a wire channel 112, each extending longitudinally through at least part of catheter body shaft 102. Imaging lumen 110 may have a bore of approximately 0.014 inches to approximately 0.020 inches, with approximately 0.016 inches being one proposed value for accommodating an OCT optical assembly with rotational clearance. Wire channel 112 may have a bore of approximately 0.016 inches to approximately 0.021 inches, with approximately 0.018 inches being one proposed value suitable for receiving a microwire 126 having an outer diameter of approximately 0.014 inches to approximately 0.018 inches. In some implementations, a 0.014-inch microwire may be used as a primary configuration, while a 0.018-inch microwire may be accommodated in selected configurations.

The monorail transition zone 104 may be disposed distal to catheter body shaft 102 and proximal to imaging zone 106. Monorail transition zone 104 may have a longitudinal length of approximately 3 mm to approximately 12 mm, with approximately 6 mm being one proposed value. Monorail transition zone 104 may provide a region where wire channel 112 terminates, transitions, opens, or otherwise communicates with an exterior of platform 100. In the illustrated configuration, monorail transition zone 104 may include a ventral relief 116, which may be disposed at or along a ventral surface of platform 100, such as at an approximately 6 o’clock position relative to the cross-sectional orientation shown in the drawings. Ventral relief 116 may provide a flush-profile contoured exit geometry, a smooth wire-exit transition, and minimal outer-diameter impact. During navigation, ventral relief 116 may function as a microwire exit location so that microwire 126 transitions from the internal wire channel 112 to an exterior monorail path. During imaging, after microwire 126 is removed or partially withdrawn, the now-empty wire channel 112 and the monorail exit may serve as a flush conduit for saline, contrast, heparinized saline, or another suitable fluid used to clear blood across the OCT imaging window. This sequential dual-function arrangement may allow the same opening to support wire exit during navigation and fluid delivery during imaging without requiring a dedicated third catheter wall penetration.

Imaging zone 106 may be disposed distal to monorail transition zone 104 and may be configured to house, support, or cooperate with an OCT imaging assembly 120. Imaging zone 106 may have a longitudinal length of approximately 15 mm to approximately 40 mm, with approximately 20 mm being one proposed value that may be used for coverage of at least part of an M1 segment or a comparable target vessel segment. Imaging zone 106 may have an outer diameter of approximately 0.045 inches to approximately 0.058 inches, with approximately 0.055 inches being one proposed value. In some implementations, the outer diameter of imaging zone 106 may remain substantially constant throughout its longitudinal length. The reduction from the larger diameter of catheter body shaft 102 to the smaller diameter of imaging zone 106 may occur at or near monorail transition zone 104. Imaging zone 106 may be configured as a single-lumen region in which imaging lumen 110 continues and wire channel 112 is not routed through imaging zone 106. In other configurations, imaging zone 106 may include one or more non-functional cavities, bonding voids, material-transition spaces, or vestigial structures, while still being configured such that microwire 126 is not routed through the OCT optical imaging field. By removing the wire channel 112 from imaging zone 106, platform 100 may provide improved optical clearance for circumferential imaging.

Distal tip zone 108 may be disposed distal to imaging zone 106 and may have a length of approximately 5 mm to approximately 20 mm, with approximately 12 mm being one proposed value. Distal tip zone 108 may have a distal terminus outer diameter of approximately 0.028 inches to approximately 0.040 inches, with dimensions selected based on the intended target anatomy and microwire compatibility. Distal tip zone 108 may include a distal taper 124 that graduates from the imaging zone diameter toward the terminus. The taper geometry may be symmetric or asymmetric. In an asymmetric configuration, the dorsal taper rate may be gentler to maintain structural continuity with the proximal catheter construction, while the ventral taper rate may be more aggressive to reduce material preferentially from the ventral surface. Such an asymmetric taper may produce a preferential ventral deflection characteristic under bending loads and may coordinate with the structural transition at monorail transition zone 104. The combined length of monorail transition zone 104, imaging zone 106, and distal tip zone 108 may define a distal working region having a total length of approximately 23 mm to approximately 72 mm, with approximately 38 mm being one proposed configuration based on a 6 mm transition zone, a 20 mm imaging zone, and a 12 mm distal tip.

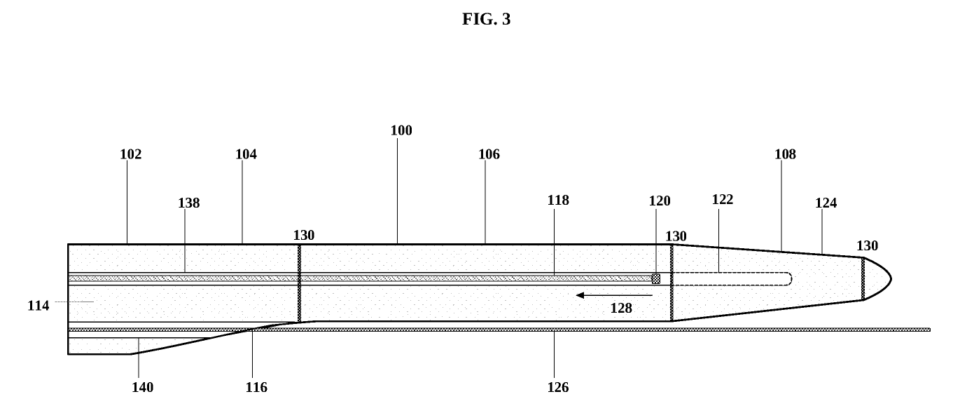

Referring now to FIG. 3, an enlarged longitudinal view of the distal portion of platform 100 illustrates the relationship among monorail transition zone 104, imaging zone 106, distal tip zone 108, imaging lumen 110, wire channel 112, ventral relief 116, OCT drive cable 118, OCT imaging assembly 120, vestigial lumen 122, microwire 126, pullback direction 128, and radiopaque marker 130. Imaging lumen 110 may extend longitudinally through catheter body shaft 102, through monorail transition zone 104, through imaging zone 106, and into at least part of distal tip zone 108. OCT drive cable 118 may extend through imaging lumen 110 and may support rotation, translation, pullback, torque transmission, optical signal transmission, or another movement or communication associated with OCT imaging assembly 120. OCT imaging assembly 120 may be positioned near a distal portion of imaging zone 106 or at another location suitable for acquiring OCT imaging data. OCT imaging assembly 120 may include an optical fiber, lens, prism, mirror, gradient-index lens, reflective surface, refractive component, focusing component, rotary optical component, or another optical component suitable for intraluminal OCT imaging. In some implementations, OCT imaging assembly 120 may operate at or near an OCT-compatible near-infrared wavelength, such as approximately 1300 nm, although other wavelengths suitable for intraluminal OCT may be used.

As shown in FIG. 3, wire channel 112 may extend through catheter body shaft 102 and may open outwardly at monorail transition zone 104. Microwire 126 may extend through wire channel 112 and then exit platform 100 through ventral relief 116 before reaching imaging zone 106. After exiting, microwire 126 may extend externally along imaging zone 106 and distal tip zone 108 in a monorail configuration. The location of the exit may be proximal to an imaging-zone boundary so that imaging zone 106 is not occupied by wire channel 112. This arrangement may reduce optical obstruction because OCT imaging relies on light transmission and may be adversely affected by adjacent lumens, polymer walls, wires, metallic structures, or other structures positioned within the imaging field. In some comparative design contexts, a side-by-side dual-lumen OCT arrangement may provide only a partial usable field of view due to an optical dead zone. The monorail architecture of platform 100 may be configured to provide substantially 360-degree circumferential optical imaging by leaving imaging zone 106 substantially free of adjacent wire-channel obstruction. This arrangement may be particularly useful for asymmetric vessel pathology, including eccentric plaque, dissection, thrombus, device malapposition, aneurysm neck features, and other localized vessel wall conditions.

Still referring to FIG. 3, one or more radiopaque markers 130 may be positioned along the distal region of platform 100. In the illustrated implementation, three radiopaque markers 130 may be positioned to provide fluoroscopic confirmation of tip position and imaging zone boundaries. For example, a first marker may be positioned near the distal tip or near a distal terminus region, a second marker may be positioned near a distal boundary of imaging zone 106, and a third marker may be positioned near a proximal boundary of imaging zone 106 or monorail transition zone 104. Marker material may include tungsten, platinum, platinum-iridium, tantalum, gold, polymer-metal blends, radiopaque filler-loaded polymer, or another fluoroscopically visible material. Pullback direction 128 may indicate a direction in which OCT imaging assembly 120, OCT drive cable 118, or a related imaging core may be moved during imaging acquisition. In some implementations, OCT imaging assembly 120 may be rotated and pulled proximally to generate a longitudinal data set or a series of cross-sectional images. Imaging zone 106 may be positioned relative to radiopaque markers 130, target site TS, and angiographic landmarks so that intraluminal OCT data may be correlated with procedural anatomy.

Vestigial lumen 122 may optionally continue from imaging zone 106 into distal tip zone 108, as shown in FIG. 3. Vestigial lumen 122 may be non-functional in that it need not carry microwire 126 and need not provide an active imaging path. When present, vestigial lumen 122 may facilitate manufacturing, bonding, junction integrity, flexibility, or navigational compliance. For example, vestigial lumen 122 may enhance junction integrity at an imaging-zone-to-tip boundary through dual-interface bonding at an inner lumen wall and an outer circumference. Vestigial lumen 122 may also permit a hollow thin-walled construction in at least part of distal tip zone 108, which may deflect more readily under bending loads than a solid structure of comparable outer diameter. In other implementations, distal tip zone 108 may be solid, filled, partially hollow, multi-durometer, coated, reinforced in selected locations, or formed without vestigial lumen 122. Distal tip zone 108 may be configured for navigation in vessels having diameters in the range of approximately 1.0 mm to approximately 5.5 mm, although the particular vessel diameter may depend on anatomy, access route, and physician judgment. In a representative target strategy, distal tip zone 108 may be parked in a mid-M2 segment or distal M2/proximal M3 region while imaging zone 106 encompasses at least part of an M1 segment and the ICA terminus.

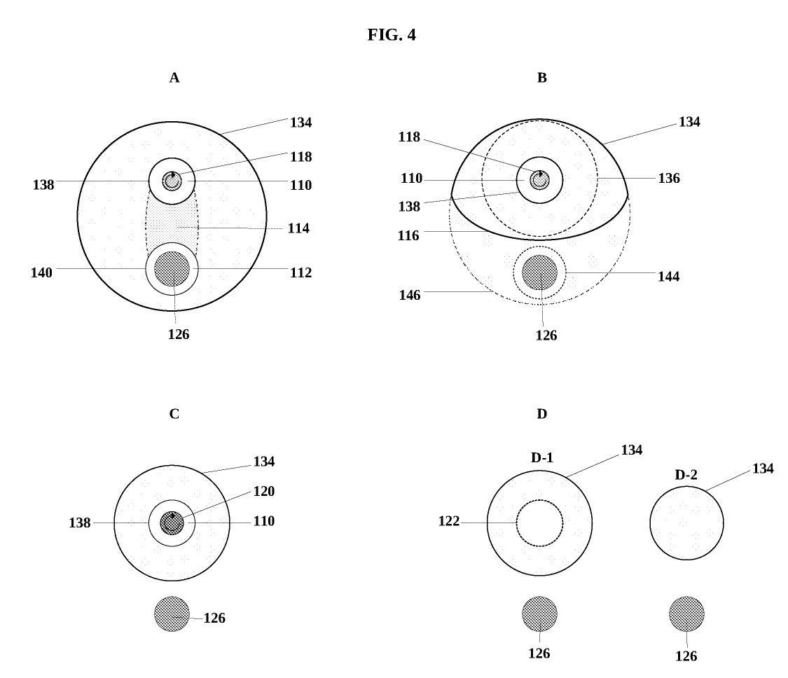

Referring now to FIG. 4, a series of cross-sectional views illustrate representative internal configurations of platform 100 through catheter body shaft 102, monorail transition zone 104, imaging zone 106, and distal tip zone 108. In a first section corresponding to catheter body shaft 102, imaging lumen 110 and wire channel 112 may be arranged in a dorsal-ventral configuration. Imaging lumen wall 138 may define or surround imaging lumen 110, and wire channel wall 140 may define or surround wire channel 112. Extrusion boundary 114 may be positioned between imaging lumen wall 138 and wire channel wall 140 and may function as a septum or structural divider. Outer polymer jacket 134 may surround imaging lumen wall 138, wire channel wall 140, and extrusion boundary 114. OCT drive cable 118 may be disposed within imaging lumen 110, and microwire 126 may be disposed within wire channel 112 during advancement. In some implementations, the cross-section may have a generally circular outer diameter with a vertically stacked dual-extrusion core. The imaging lumen 110 may be positioned dorsally at approximately a 12 o’clock position, while the wire channel 112 may be positioned ventrally at approximately a 6 o’clock position. This geometry may preserve over-the-wire navigation proximally while setting up a controlled ventral exit of microwire 126 at monorail transition zone 104.

A second section in FIG. 4 may correspond to monorail transition zone 104. In this region, the catheter body may transition from a dual-lumen or dual-channel configuration toward a reduced-profile geometry suitable for imaging zone 106. Ventral relief 116 may be defined by a lower-wall contour that opens the wire channel 112 to the exterior. Microwire 126 may be shown outside the main catheter body after exiting through ventral relief 116. Imaging lumen 110 may remain within the catheter body and may continue toward imaging zone 106. Section C OD reference profile 136 and other reference profiles may illustrate the change from a larger proximal catheter body profile toward the smaller imaging-zone profile. The transition geometry may maintain structural integrity of outer polymer jacket 134 while allowing the wire path to change from internal to external. Ventral relief 116 may be shaped to avoid a sharp edge or abrupt ledge at the wire exit. In some implementations, the outer diameter impact of the wire exit may be negligible because the 0.014-inch to 0.018-inch microwire may exit from the ventral surface of a catheter body having an outer diameter in the range of approximately 0.060 inches to approximately 0.072 inches.

A third section in FIG. 4 may correspond to imaging zone 106. In this region, imaging lumen 110 may be centered or substantially centered within outer polymer jacket 134 to provide circumferential optical clearance. OCT imaging assembly 120 may be disposed within imaging lumen 110 and may be configured for rotational imaging. Microwire 126 may be positioned outside the catheter body in a monorail relationship. Wire channel 112 may be absent from imaging zone 106, may terminate proximal to imaging zone 106, or may not be routed through the optical imaging field. The outer polymer jacket 134 at imaging zone 106 may comprise an optically transmissive polymer segment compatible with OCT wavelengths, including wavelengths around approximately 1300 nm. The polymer material in imaging zone 106 may comprise low-durometer Pebax, cyclic olefin polymer, cyclic olefin copolymer, fluoropolymer, polyimide, or another material selected for optical transmission, catheter flexibility, bondability, and neurovascular compatibility. Reinforcement in imaging zone 106 may be non-metallic, minimal, interrupted, or absent to avoid optical interference. In one example, a distal portion of approximately 30 mm may be non-braided to reduce interference with optical transmission, while more proximal sections may use braid or coil-braid reinforcement to provide pushability and trackability.

A fourth group of sections in FIG. 4 may correspond to distal tip zone 108. In one section, vestigial lumen 122 may be depicted within a proximal portion of distal tip zone 108, while microwire 126 remains outside the catheter body. In another section, a terminal tip portion may be depicted as solid or substantially solid polymer. The distal tip may taper from an imaging zone outer diameter of approximately 0.045 inches to approximately 0.058 inches toward a distal terminus outer diameter of approximately 0.028 inches to approximately 0.040 inches. The taper may be configured to reduce vessel trauma, improve trackability, and maintain sufficient column support during advancement. The distal tip material may comprise Pebax, polyurethane, nylon, silicone, another soft polymer, or a blend or layered structure. The distal tip zone 108 may be unreinforced or minimally reinforced, and may have a lubricious or hydrophilic coating. Section A OD reference profile 146 and wire channel OD reference profile 144 may illustrate relative diameter relationships and design references. These reference profiles may be illustrative only and may be varied based on intended vessel size, imaging assembly size, and manufacturing tolerances.

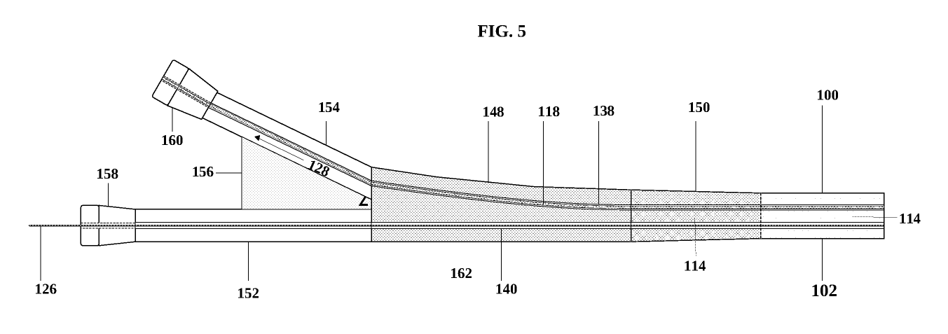

Referring now to FIG. 5, the proximal hub assembly of platform 100 may include hub body 148, strain relief 150, working port 152, imaging port 154, webbing zone 156, working port luer assembly 158, and imaging port luer assembly 160. Hub body 148 may be coupled to catheter body shaft 102 through strain relief 150. Strain relief 150 may have a length of approximately 15 mm to approximately 35 mm, with approximately 25 mm being one proposed value. Hub body 148 may have a length of approximately 30 mm to approximately 70 mm, with approximately 60 mm being one proposed value. In some implementations, hub body 148 may taper continuously from a smaller diameter near the strain relief junction, such as approximately 2.5 mm, toward a larger bifurcation region, such as approximately 5.5 mm. Working port 152 may have a length of approximately 25 mm to approximately 80 mm, with approximately 50 mm being one proposed value. Imaging port 154 may also have a length of approximately 25 mm to approximately 80 mm, with approximately 50 mm being one proposed value. In some configurations, working port 152 may be longer than imaging port 154 to maintain clearance from an operator’s working plane for wire and flush manipulation, while imaging port 154 may be angled to keep an imaging console connection away from the procedural workspace.

Working port 152 may be in communication with wire channel 112 and may provide sequential access to wire channel 112. During navigation, microwire 126 may be inserted and manipulated through working port luer assembly 158, through working port 152, and into wire channel 112 for over-the-wire guidance. During imaging, after microwire 126 is removed or withdrawn from wire channel 112, flush media may be delivered through working port 152 into the emptied wire channel 112 for blood clearance at monorail transition zone 104 and across imaging zone 106. Flush media may comprise saline, heparinized saline, contrast, dextran, or another fluid suitable for clearing blood or improving OCT optical clarity. In some implementations, a saline flush rate of approximately 4 mL/s to approximately 7 mL/s may be used as an example range for blood clearance, although rates may vary with vessel size, imaging protocol, access catheter arrangement, and clinical judgment. Imaging port 154 may branch from hub body 148 at an angle of approximately 15 degrees to approximately 60 degrees, with approximately 30 degrees being one proposed value. Imaging port 154 may provide a dedicated connection for an OCT optical fiber assembly, an optical drive unit, a controller, a data path, or another external OCT system.

Webbing zone 156 may provide a structural connection between working port 152 and imaging port 154 within the bifurcation region. Webbing zone 156 may be triangular, curved, planar, ribbed, thickened, thinned, molded, overmolded, bonded, or otherwise configured to maintain spacing and structural relationship between the port assemblies. In some implementations, a webbing zone may have a length or extent of approximately 30 mm, such as approximately 15 mm into the hub body and approximately 15 mm into the port region, although other dimensions may be used. Hub body septum 162 may separate the diverging lumen paths within hub body 148. Hub body septum 162 may be constructed as a continuation of extrusion boundary 114, an integral molded feature of hub body 148, a discrete seated element, a bonded insert, a shaped metallic spine, or another separator. Hub body 148 may house a bifurcation region in which imaging lumen 110 diverges dorsally toward imaging port 154 and wire channel 112 continues or transitions ventrally or axially toward working port 152. The hub extension beyond the catheter shaft may be approximately 80 mm to approximately 140 mm, with approximately 110 mm being one proposed value. Total device length may fall within approximately 155 cm to approximately 170 cm, depending on shaft length and hub extension.

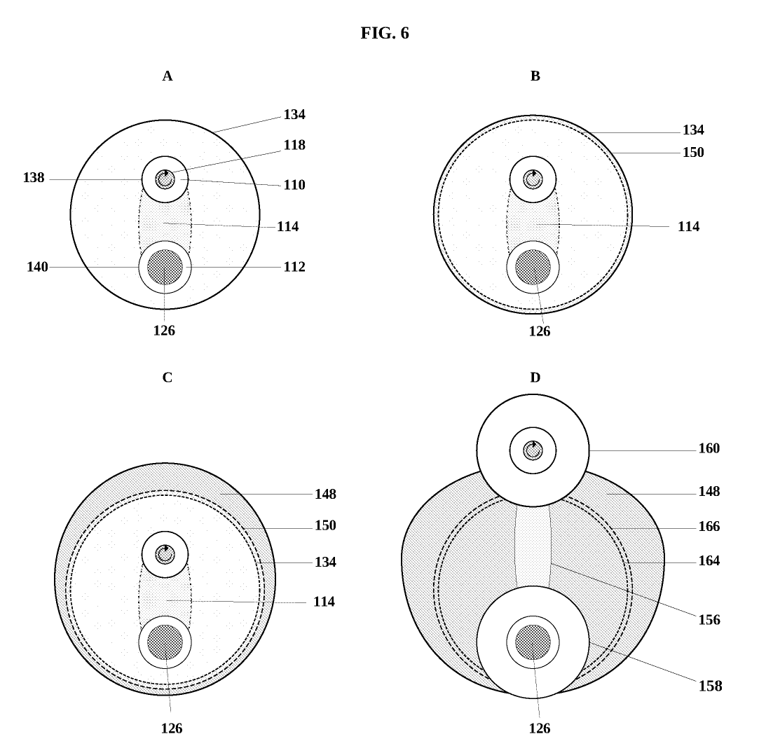

Referring now to FIG. 6, representative cross-sectional views of the proximal hub assembly may illustrate lumen routing through catheter body shaft 102, strain relief 150, hub body 148, working port 152, and imaging port 154. Catheter body OD reference profile 164 may represent the catheter body perimeter at its nominal diameter, and strain relief OD reference profile 166 may represent the strain relief perimeter at its nominal diameter. Imaging lumen wall 138 and wire channel wall 140 may continue through at least part of strain relief 150 and into hub body 148. Within hub body 148, imaging lumen 110 may migrate or curve toward imaging port 154, while wire channel 112 may remain aligned with or transition toward working port 152. OCT drive cable 118 may be routed through imaging lumen 110 and imaging port 154. Microwire 126 may be routed through wire channel 112 and working port 152 during loading, advancement, or withdrawal. The cross-sectional views may show that hub body 148 expands beyond catheter body shaft 102 and strain relief 150 at the port bifurcation. Working port luer assembly 158 may be positioned at a ventral or lower aspect, and imaging port luer assembly 160 may be positioned at a dorsal or upper aspect. Webbing zone 156 may connect the port bodies where they diverge from hub body 148.

The cross-sectional structure of FIG. 6 may also illustrate that the proximal hub assembly preserves distinct paths for imaging and wire/flush functions. Imaging lumen 110 may remain separated from wire channel 112 by hub body septum 162 or related divider structures. The imaging port luer assembly 160 may house or guide OCT drive cable 118 and may interface with an external OCT system. The working port luer assembly 158 may house or guide microwire 126 and may also serve as a connection for flushing after wire removal. Strain relief 150 may provide a graduated mechanical transition between the relatively flexible catheter body shaft 102 and the relatively rigid hub body 148. The proximal hub assembly may be molded, machined, printed, assembled from separate parts, bonded, welded, overmolded, or otherwise manufactured. Seals, valves, gaskets, rotating interfaces, compression fittings, luer connectors, hemostasis features, strain-relief sleeves, and external labels may be incorporated into the hub assembly. The hub assembly may therefore support device preparation, wire handling, fluid delivery, imaging console connection, and intra-procedural manipulation.

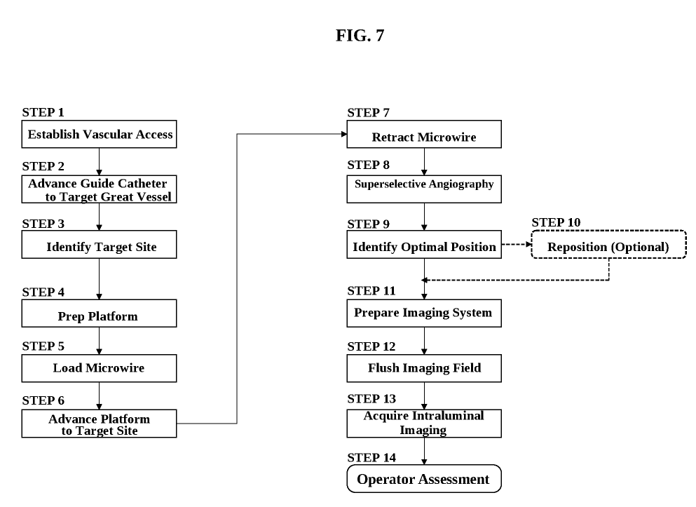

Referring now to FIG. 7, a workflow diagram may illustrate a method of using platform 100. The method may begin by establishing vascular access with access sheath A and advancing guide catheter B through access sheath A toward a target great vessel. Target site TS may be identified using fluoroscopy, angiography, digital subtraction angiography, pre-procedural imaging, intraprocedural imaging, or another localization technique. Platform 100 may be prepared by flushing one or more lumens, connecting working port luer assembly 158 to a flush source or guidewire accessory, connecting imaging port luer assembly 160 to an OCT imaging system, and loading microwire 126 through wire channel 112. Platform 100 may be advanced through guide catheter B toward target site TS while microwire 126 provides wire-guided navigation. Microwire 126 may exit platform 100 through ventral relief 116 at monorail transition zone 104 and may extend externally along imaging zone 106 and distal tip zone 108. Imaging zone 106 may then be positioned at or near target site TS. Once positioned, OCT imaging assembly 120 may acquire circumferential intraluminal imaging data while rotating, translating, pulling back, or otherwise moving relative to the vessel.

During the workflow of FIG. 7, imaging data may be used to assess vessel morphology, vessel pathology, stenosis, thrombus, dissection, aneurysm neck configuration, vessel diameter, device apposition, residual stenosis, vessel remodeling, vasospasm, vasculitis, or another intraluminal feature. Imaging data may be displayed in real time, stored, processed, co-registered with angiography, segmented, measured, annotated, or used to guide procedural decisions. In some implementations, imaging zone 106 may cover an M1 segment having a length of approximately 20 mm and a mean diameter range of approximately 2.3 mm to approximately 2.7 mm, with approximately 2.47 mm being an example DSA mean. Imaging zone 106 may also be used to assess an ICA terminus region, which may have a length of approximately 2 mm, or part of a supraclinoid ICA C6-C7 segment, which may have a length of approximately 25 mm. In some workflows, distal tip zone 108 may be parked in a mid-M2 segment or distal M2/proximal M3 region while imaging zone 106 spans M1 and ICA-terminus anatomy. The workflow may be repeated, modified, or combined with therapeutic device placement, angioplasty, stenting, thrombectomy, flow diversion, intrasaccular treatment, or other neurovascular intervention as clinically appropriate.

Referring now to FIG. 8, an anatomical system overview is shown for another implementation of an intraluminal imaging delivery assist platform 200 deployed through a neurovascular access pathway toward target site TS. Access sheath A may be positioned percutaneously through a femoral access location, and guide catheter B may be advanced through access sheath A, the iliac artery, the aorta, the aortic arch, and into a target great vessel, such as a carotid artery or vertebral artery. Platform 200 may be advanced through guide catheter B into intracranial or extracranial vasculature toward target site TS. Target site TS may comprise a location within the internal carotid artery, middle cerebral artery, vertebrobasilar system, or another neurovascular territory. Target site TS may be associated with stenosis, thrombus, aneurysm, previously implanted device, dissection, vasospasm, vasculitis, vessel narrowing, vessel sizing, or another diagnostic or procedural concern. Platform 200 may be advanced to target site TS over microwire 226 in a full-length over-the-wire configuration. Unlike platform 100, which may transition to a monorail configuration before the imaging zone, platform 200 may maintain microwire 226 within a working lumen through the catheter body shaft, imaging zone 206, and distal tip zone during navigation.

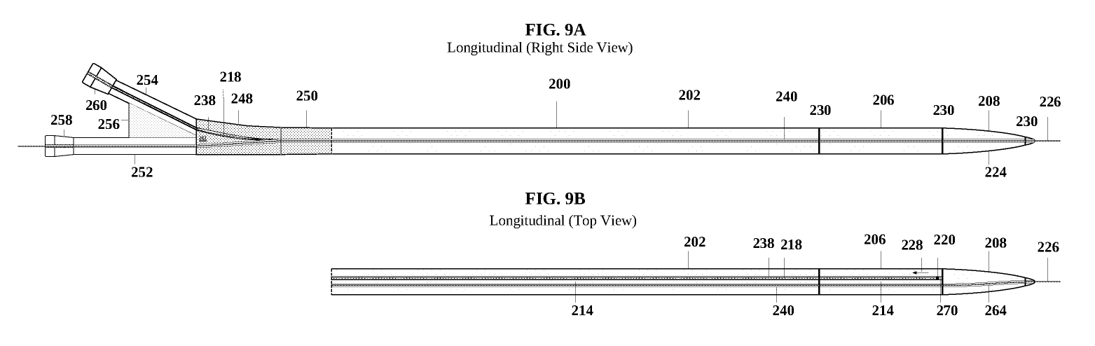

Referring now to FIGS. 9A and 9B, platform 200 may include catheter body shaft 202, imaging zone 206, distal tip zone 208, IVUS drive cable 218, IVUS imaging assembly 220, distal taper 224, microwire 226, pullback direction 228, radiopaque marker 230, and a proximal hub assembly. FIG. 9A may provide a longitudinal side view, and FIG. 9B may provide a longitudinal top view illustrating the dual-lumen arrangement through catheter body shaft 202 and imaging zone 206. Platform 200 may be configured for a working length of approximately 140 cm to approximately 170 cm, and in one proposed configuration may include a shaft length of approximately 155 cm. When a hub extension of approximately 110 mm, or approximately 11 cm, is used, the total device length may be approximately 166 cm. Catheter body shaft 202 may have an outer diameter of approximately 0.060 inches to approximately 0.072 inches, with approximately 0.065 inches being one proposed proximal shaft value. In a mid-shaft to imaging housing region, platform 200 may have an outer diameter of approximately 0.062 inches, corresponding to approximately 1.575 mm. At or near an M2 marker or transition region, the outer diameter may be approximately 0.051 inches, corresponding to approximately 1.30 mm. Distal tip zone 208 may taper toward an outer diameter of approximately 0.035 inches, corresponding to approximately 0.889 mm, in one proposed configuration.

Platform 200 may use a horizontal oval-core dual-lumen architecture. Imaging lumen wall 238 and working lumen wall 240 may be arranged side-by-side within a continuous outer polymer jacket 266. The working lumen defined by working lumen wall 240 may have a bore of approximately 0.016 inches to approximately 0.021 inches, with approximately 0.018 inches, or approximately 0.457 mm, being one proposed value for a 0.014-inch microwire. The imaging lumen defined by imaging lumen wall 238 may have a bore of approximately 0.014 inches to approximately 0.020 inches, with approximately 0.016 inches, or approximately 0.406 mm, being one proposed value for an IVUS drive cable and PZT transducer. In one configuration, the dual-lumen core may define an oval envelope of approximately 0.964 mm by approximately 0.457 mm, corresponding to approximately 0.038 inches by approximately 0.018 inches. A radial clearance of approximately 0.305 mm, or approximately 0.012 inches, may be provided around the oval core. An internal web or extrusion boundary 214 between the lumens may have a thickness of approximately 0.076 mm, or approximately 0.003 inches, in one representative configuration. These values may be modified based on transducer size, working lumen requirements, manufacturing tolerances, and acoustic performance.

Imaging zone 206 may have a length of approximately 15 mm to approximately 40 mm in a broad implementation. In one IVUS-specific proposed configuration, an imaging housing length may be approximately 47.5 mm, with a usable imaging zone of up to approximately 37.5 mm, a proximal buffer zone of approximately 5 mm, and a distal buffer zone of approximately 5 mm. Distal tip zone 208 may have a length of approximately 5 mm to approximately 20 mm in a broad implementation. In one proposed IVUS configuration, distal tip zone 208 may have a tapered tip length of approximately 7.5 mm and a total taper length of approximately 12.5 mm, including a 5 mm distal buffer and a 7.5 mm tip. The distal outer diameter may taper from approximately 0.062 inches to approximately 0.035 inches over approximately 12.5 mm, producing an example taper gradient of approximately 0.00216 inches per mm. Imaging zone 206 may have an outer diameter of approximately 0.045 inches to approximately 0.060 inches, and may maintain a constant or substantially constant outer diameter throughout the imaging region. Radiopaque markers 230 may be positioned near boundaries of imaging zone 206, near distal tip zone 208, or at anatomical reference points to facilitate fluoroscopic positioning.

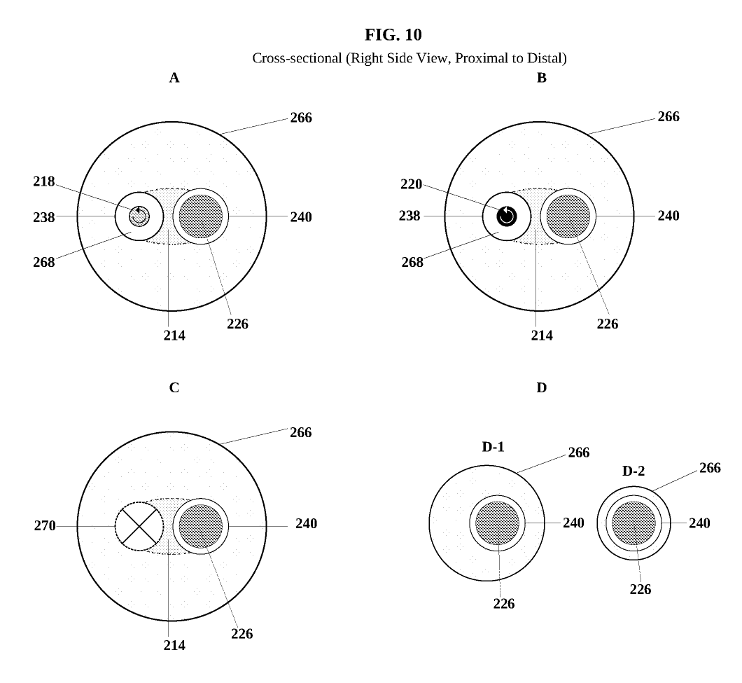

Referring now to FIG. 10, a series of cross-sectional views illustrate internal configurations of platform 200 through catheter body shaft 202, imaging zone 206, and distal tip zone 208. A first section may show imaging lumen wall 238 and working lumen wall 240 arranged laterally adjacent to one another in a side-by-side orientation. Working lumen wall 240 may define a working lumen that receives microwire 226. Imaging lumen wall 238 may define an imaging lumen that receives IVUS drive cable 218 and acoustic coupling medium 268. Extrusion boundary 214 may separate the imaging lumen wall 238 from working lumen wall 240. Outer polymer jacket 266 may surround the dual-lumen core and may define the outer surface of platform 200. The side-by-side oval-core arrangement may provide quasi-centric positioning of the dual-lumen pair within the circular outer diameter while preserving radial clearance for polymer jacket material, reinforcement, acoustic transmission, and structural support. The architecture may be selected because IVUS uses acoustic energy rather than optical energy, and acoustic waves may tolerate the presence of an adjacent working lumen when acoustic coupling media and suitable signal processing are used.

Another section in FIG. 10 may show IVUS imaging assembly 220 disposed within the imaging lumen. IVUS imaging assembly 220 may include a PZT transducer, rotating ultrasound transducer, capacitive micromachined ultrasonic transducer, piezoelectric micromachined ultrasonic transducer, transducer array, or another intravascular ultrasound component. IVUS drive cable 218 may rotate or otherwise actuate IVUS imaging assembly 220 to acquire circumferential ultrasound data. Acoustic coupling medium 268 may fill or partially fill the imaging lumen around IVUS drive cable 218 and IVUS imaging assembly 220. Acoustic coupling medium 268 may include saline, heparinized saline, contrast, another biocompatible liquid, or another medium suitable for acoustic transmission. During navigation mode, microwire 226 may remain within the working lumen. During imaging mode, microwire 226 may be withdrawn through the proximal hub assembly, and acoustic coupling medium 268 may be delivered into the working lumen through working port 252 so that both the imaging lumen and the working lumen may contribute to acoustic coupling. This dual acoustic coupling pathway may reduce transmission loss or acoustic interference caused by the adjacent working lumen.

A further section in FIG. 10 may show imaging lumen termination 270 at or near a distal boundary of imaging zone 206. At this location, the imaging lumen may terminate while the working lumen continues into distal tip zone 208. The working lumen may undergo a working lumen centration transition 264, in which the working lumen gradually moves from a laterally offset position toward a centered or more centered position at the distal tip terminus. The working lumen centration transition 264 may be used to maintain over-the-wire navigation while reducing the distal profile and improving distal tip behavior. The asymmetric material redistribution caused by the lateral-to-centered transition may influence bending, tracking, and deflection behavior under load. Distal tip zone 208 may include distal taper 224, which may reduce the outer diameter toward the distal terminus. In one anatomical positioning example, a tapered tip having an outer diameter of approximately 0.889 mm may be positioned in a mid-M2 vessel having a diameter of approximately 1.2 mm to approximately 1.5 mm, corresponding to a lumen occupation of approximately 59% to approximately 74%. An M2 marker region having an outer diameter of approximately 1.30 mm may align with an M1-M2 junction in a vessel diameter of approximately 2.3 mm to approximately 2.7 mm, corresponding to a lumen occupation of approximately 48% to approximately 57%. An imaging housing having an outer diameter of approximately 1.575 mm may be positioned in an M1 segment having a diameter of approximately 2.3 mm to approximately 2.7 mm, corresponding to a lumen occupation of approximately 58% to approximately 68%.

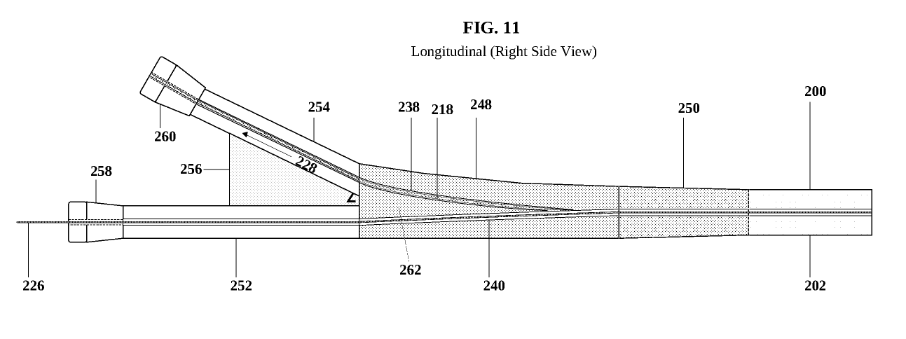

Referring now to FIG. 11, the proximal hub assembly of platform 200 may include hub body 248, strain relief 250, working port 252, imaging port 254, webbing zone 256, working port luer assembly 258, and imaging port luer assembly 260. Strain relief 250 may provide a graduated mechanical transition between catheter body shaft 202 and hub body 248. Strain relief 250 may have a length of approximately 15 mm to approximately 35 mm, with approximately 25 mm being one proposed value. In one configuration, strain relief 250 may taper from approximately 1.575 mm to approximately 2.5 mm over approximately 25 mm, corresponding to an approximate gradient of 0.037 mm/mm. Hub body 248 may have a length of approximately 30 mm to approximately 70 mm, with approximately 60 mm being one proposed value. Hub body 248 may transition from approximately 2.5 mm at a strain relief junction to approximately 5.5 mm at a bifurcation region. Working port 252 may have a length of approximately 25 mm to approximately 80 mm, with approximately 50 mm being one proposed value. Working port 252 may have an outer diameter of approximately 4 mm at a bifurcation exit and may taper to approximately 5 mm at a luer collar, with a bore of approximately 0.018 inches. Imaging port 254 may have a length of approximately 25 mm to approximately 80 mm, with approximately 50 mm being one proposed value, and may have an outer diameter of approximately 4 mm at a bifurcation exit with a bore of approximately 0.016 inches.

Imaging port 254 may branch from hub body 248 at an angle of approximately 15 degrees to approximately 60 degrees, with approximately 30 degrees being one proposed value. Working port 252 may be oriented axially or substantially axially. Webbing zone 256 may provide a structural connection between working port 252 and imaging port 254 within the bifurcation region. In one representative configuration, webbing zone 256 may extend approximately 30 mm, including approximately 15 mm into hub body 248 and approximately 15 mm into the port region. Hub body septum 262 may separate diverging lumen paths within hub body 248. In the illustrated configuration, working lumen wall 240 may continue axially toward working port 252 and may optionally translate ventrally to accommodate the diverging hub geometry. Imaging lumen wall 238 may transition through a curved path toward the angled orientation of imaging port 254. IVUS drive cable 218 may route through imaging lumen wall 238, through hub body 248, and out through imaging port luer assembly 260 to an external IVUS console, drive motor, image processing unit, or other external component. Acoustic coupling medium 268 may fill imaging lumen wall 238 along the routed path to maintain acoustic coupling continuity between the hub assembly and IVUS imaging assembly 220.

Working port 252 may serve sequential dual functions in platform 200. During navigation mode, microwire 226 may be inserted through working port luer assembly 258 and into the working lumen defined by working lumen wall 240. Microwire 226 may then extend through catheter body shaft 202, imaging zone 206, distal tip zone 208, and beyond the distal tip to guide platform 200. During imaging mode, microwire 226 may be retracted through working port luer assembly 258, leaving the working lumen available for acoustic coupling medium delivery. Acoustic coupling medium 268 may be delivered through working port 252 into the working lumen to improve acoustic transmission through the adjacent lumen region. This sequential use of working port 252 may avoid the need for a dedicated third port while still permitting guidewire access and coupling medium delivery. Alternative hub configurations may include additional ports, separate flush ports, separate coupling ports, hemostasis valves, rotating seals, adapters, or other components. The working port 252 and imaging port 254 may be arranged to reduce procedural clutter, maintain user access, and preserve a manageable connection geometry near the sterile field.

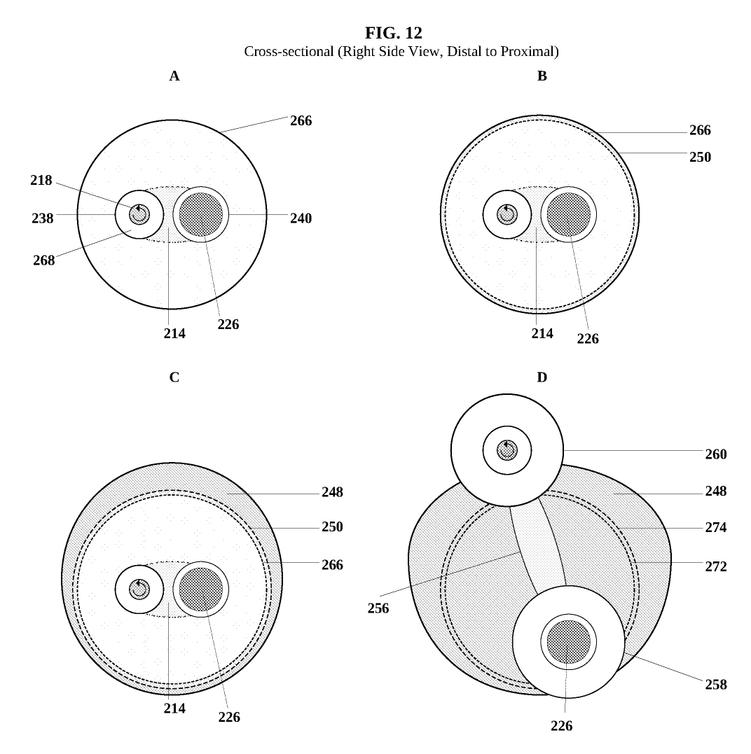

Referring now to FIG. 12, representative cross-sectional views of the proximal hub assembly of platform 200 may illustrate lumen routing through catheter body shaft 202, strain relief 250, hub body 248, working port 252, and imaging port 254. Catheter body reference profile 272 may represent the catheter body perimeter at its nominal diameter, and strain relief reference profile 274 may represent the strain relief perimeter at its nominal diameter. In a catheter-body section, imaging lumen wall 238 and working lumen wall 240 may remain side-by-side within outer polymer jacket 266. IVUS drive cable 218 and acoustic coupling medium 268 may be disposed within imaging lumen wall 238, while microwire 226 may be disposed within working lumen wall 240 during navigation. In a strain-relief section, the dual-lumen oval-core arrangement may remain present, while an outer strain relief profile surrounds the catheter body profile. In a hub body section, the lumen paths may begin to separate while hub body septum 262 maintains separation between the imaging path and working path. In a port-bifurcation section, imaging port luer assembly 260 may be positioned at a dorsal aspect and working port luer assembly 258 may be positioned at a ventral aspect, with webbing zone 256 connecting the two port bodies along a diagonal or angled relationship.

The cross-sectional views of FIG. 12 may show that platform 200 uses independent migration of lumen paths through the hub assembly while preserving the individual function of each lumen. Imaging lumen wall 238 may translate dorsally toward imaging port 254 while maintaining a path for IVUS drive cable 218 and acoustic coupling medium 268. Working lumen wall 240 may continue toward working port 252 while maintaining a path for microwire 226 and later for acoustic coupling medium delivery. Hub body 248 may expand beyond the catheter body and strain relief boundaries at the port bifurcation. Hub body septum 262 may be formed as a continuation of extrusion boundary 214, an integral molded hub feature, a discrete insert, a bonded element, or another separator. The port assemblies may include luer connectors, hemostasis valves, seals, rotating drive interfaces, compression fittings, caps, flush connectors, or external adapters. The cross-sectional arrangement may support preparation, wire manipulation, imaging connection, coupling medium delivery, and withdrawal without merging the imaging and working pathways.

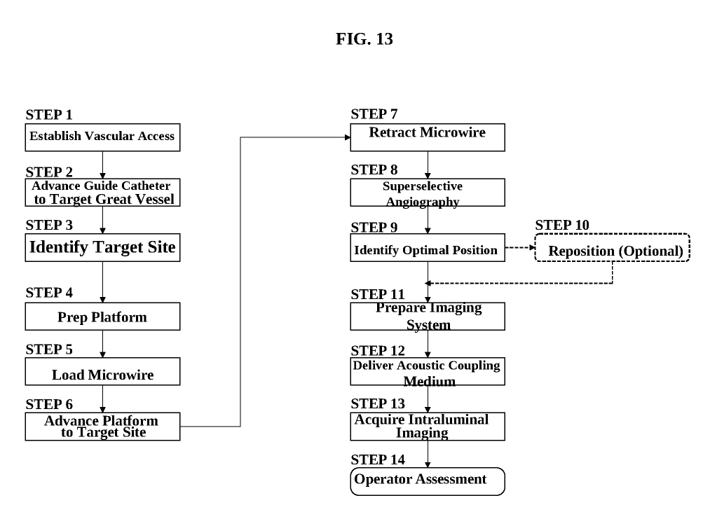

Referring now to FIG. 13, a workflow diagram may illustrate a method of using platform 200 for intravascular ultrasound imaging and delivery assistance. The method may begin by establishing vascular access with access sheath A and advancing guide catheter B toward a target great vessel. Target site TS may be identified by fluoroscopy, angiography, digital subtraction angiography, pre-procedural imaging, intraprocedural imaging, or another localization technique. Platform 200 may be prepared by connecting a flush or coupling medium source to working port luer assembly 258, connecting an IVUS imaging system to imaging port luer assembly 260, and loading microwire 226 through working port 252 and the working lumen. Platform 200 may be advanced over microwire 226 through guide catheter B toward target site TS. In navigation mode, microwire 226 may remain within working lumen wall 240 through catheter body shaft 202, imaging zone 206, and distal tip zone 208. Once imaging zone 206 is positioned at or near target site TS, microwire 226 may be withdrawn through working port luer assembly 258. Acoustic coupling medium 268 may then be introduced through working port 252 to fill or partially fill the working lumen and improve acoustic transmission across the dual-lumen construction.

During IVUS imaging with platform 200, IVUS imaging assembly 220 may acquire circumferential or substantially circumferential ultrasound imaging data from imaging zone 206. IVUS drive cable 218 may rotate, translate, pull back, advance, or otherwise move according to a selected imaging protocol. Acoustic coupling medium 268 within the imaging lumen may provide acoustic coupling around IVUS imaging assembly 220, while acoustic coupling medium 268 within the working lumen may mitigate acoustic shadowing or transmission loss caused by the adjacent working lumen. IVUS frequencies may be approximately 40 MHz to approximately 60 MHz in some implementations, although other frequencies may be used depending on desired penetration depth, resolution, transducer size, and neurovascular target anatomy. The imaging data may be transmitted through imaging port 254 to an IVUS imaging console, processing module, display, or external system. The data may be used to evaluate vessel diameter, stenosis, plaque, thrombus, vessel wall morphology, dissection, aneurysm-related anatomy, vasospasm, device apposition, treatment effects, or other neurovascular conditions. After imaging, platform 200 may be repositioned, withdrawn, used for additional imaging passes, or used to inform subsequent intervention.

The OCT implementation of platform 100 and the IVUS implementation of platform 200 may be understood as separate imaging-specific embodiments of a broader intraluminal imaging delivery assist platform. Platform 100 may use a hybrid over-the-wire-to-monorail architecture to address optical imaging requirements. Platform 200 may use a full-length over-the-wire dual-lumen architecture to preserve guidewire access and exploit acoustic transmission characteristics of ultrasound. Both platforms may be configured for internal carotid artery and middle cerebral artery navigation, including M1 and M2 segment access, while also being suitable for other neurovascular territories. In one anatomical coverage strategy for platform 200, distal tip zone 208 may be inserted approximately 10 mm to approximately 15 mm into an M2 segment, imaging zone 206 may cover an M1 segment having an approximate mean length of 20 mm, the ICA terminus may be within the imaging window, and a supraclinoid ICA C6-C7 segment of approximately 25 mm may be partially covered depending on pullback length. In some examples, a usable IVUS imaging window of approximately 37.5 mm may cover approximately 100% of the M1 segment and ICA terminus and approximately 62% of the supraclinoid ICA C6-C7 segment. These anatomical references may be used to guide dimensional selection, but are not limiting.

The materials and construction of platforms 100, 200 may vary according to modality, target anatomy, manufacturing process, and clinical workflow. Catheter body shafts 102, 202 may be formed from Pebax, polyimide, polyurethane, nylon, polyethylene, fluoropolymers, cyclic olefin polymer, cyclic olefin copolymer, silicone, PDMS, or combinations thereof. Lumen liners may comprise PTFE, FEP, HDPE, lubricious polymer, hydrophilic material, or another low-friction material. Reinforcement may comprise stainless steel braid, nitinol braid, polymer braid, coil, hybrid coil-braid, cut hypotube, laser-cut hypotube, non-metallic reinforcement, fiber reinforcement, composite reinforcement, or another support structure. In platform 100, the proximal section may use stainless steel braid for pushability and torque transmission, an intermediate section may use coil-braid transition construction for flexibility, imaging zone 106 may use non-metallic or minimal reinforcement to avoid optical interference, and distal tip zone 108 may use a softer unreinforced polymer. In platform 200, reinforcement may be selected to preserve acoustic transmission through imaging zone 206 while maintaining pushability and flexibility. Either platform may include hydrophilic coating over a length of approximately 90 cm in one primary configuration, or approximately 60 cm in an alternative cost-reduction or design variant. Other coating lengths may be used based on access anatomy and desired lubricity.

The platforms 100, 200 may be used in connection with a range of neurovascular pathologies and procedures. In ischemic stroke associated with large or medium vessel occlusion, the platforms may help characterize occlusion, thrombus, vessel wall condition, post-recanalization injury, or residual narrowing. In aneurysm procedures, the platforms may assist with assessing aneurysm neck configuration, parent vessel diameter, branch incorporation, flow diverter apposition, intrasaccular device relationship, residual neck, or post-treatment vessel status. In intracranial atherosclerotic disease, the platforms may assist with stenosis quantification, plaque characterization, vessel remodeling assessment, angioplasty planning, stent sizing, or post-stent assessment. In dissection, vasospasm, vasculitis, or traumatic vascular injury, the platforms may assist with identifying wall changes, luminal compromise, flap configuration, vessel narrowing, or treatment response. In implanted-device assessment, the platforms may be used to evaluate stent apposition, flow diverter wall contact, thrombus, neointimal coverage, residual stenosis, or vessel remodeling. These use cases are illustrative and may be combined with diagnostic imaging, intervention planning, peri-procedural assessment, and post-treatment evaluation.

The external imaging systems associated with platforms 100, 200 may vary. Platform 100 may connect to an OCT console through imaging port 154, and the OCT console may include an optical source, interferometer, rotary drive, pullback motor, signal processor, image processor, display, storage unit, and user interface. Platform 200 may connect to an IVUS console through imaging port 254, and the IVUS console may include a drive motor, ultrasound signal generator, receiver, image processor, display, storage unit, and user interface. Either system may provide real-time images, cross-sectional images, longitudinal reconstructions, pullback maps, measurements, annotations, automated border detection, vessel diameter measurements, lesion measurements, device apposition analysis, or co-registration with angiography. Either system may also incorporate wired or wireless data transmission, artificial-intelligence-assisted image interpretation, machine-learning segmentation, automated plaque or thrombus characterization, device apposition quantification, stenosis measurement, or other software-based analysis. Such features may be used as optional enhancements and need not be present in every implementation.

Although FIGS. 1-7 depict platform 100 as an OCT hybrid monorail implementation and FIGS. 8-13 depict platform 200 as an IVUS dual-lumen over-the-wire implementation, selected features may be interchanged or adapted where feasible. A hub feature described with respect to platform 100 may be used with platform 200 if lumen routing is adapted. A coating, radiopaque marker arrangement, stiffness gradient, access catheter compatibility range, material selection, or distal tip construction described with respect to one platform may be used with the other platform. Platform 100 may be modified to include additional flushing paths, alternative wire-exit geometries, different ventral relief shapes, alternative monorail exit angles, different distal tip constructions, or different OCT optical assemblies. Platform 200 may be modified to include different oval-core proportions, different working lumen dimensions, different imaging lumen dimensions, different acoustic coupling media, additional hub ports, alternative transducer technologies, or alternative working lumen centration transitions. In another implementation, an IVUS imaging system may be adapted into the monorail architecture of platform 100 by replacing OCT imaging assembly 120 with an IVUS transducer and selecting acoustically transmissive imaging-zone materials. In a further implementation, a combined OCT and IVUS architecture may be used if a catheter envelope, imaging-zone design, and workflow can accommodate both modalities.

The dimensions, ranges, and proposed values described herein may be approximate and may be subject to engineering optimization, prototype validation, manufacturing tolerances, material selection, imaging component size, access catheter compatibility, and clinical workflow requirements. The particular dimensions are provided to support the disclosed embodiments and to describe representative configurations, not to limit the invention to exact numerical values. Values may vary by plus or minus manufacturing tolerances, by design families, by target vessel size, by imaging modality, or by intended procedure. The dimensions may also be scaled for pediatric, adult, intracranial, extracranial, carotid, vertebrobasilar, coronary, peripheral, or other vascular applications if desired. The reference numerals used herein are provided to facilitate understanding of the drawings and do not limit the claimed subject matter to the illustrated structures. Any feature described in connection with one embodiment may be used with, substituted for, or combined with a feature described in connection with another embodiment unless the context indicates otherwise. Since many modifications, variations, and changes in detail can be made to the described embodiments, all matters in the foregoing description and shown in the accompanying drawings should be interpreted as illustrative and not in a limiting sense. The scope of the invention should be determined by the appended claims and their legal equivalents.

What is claimed is:

1. An intraluminal imaging delivery assist platform for neurovascular use, comprising:
a catheter shaft configured for advancement through a neurovascular guide catheter, the catheter shaft including an imaging lumen and a working lumen arranged side-by-side within at least a portion of the catheter shaft;
a proximal hub assembly coupled to the catheter shaft, the proximal hub assembly including a working port in communication with the working lumen and an imaging port in communication with the imaging lumen;
an intravascular ultrasound imaging assembly disposed at least partially within the imaging lumen and configured to acquire circumferential intraluminal ultrasound imaging data from an imaging region of the catheter shaft;
a distal tip region disposed distal to the imaging region and configured for atraumatic advancement through neurovascular anatomy; and
a microwire receivable within the working lumen during advancement of the catheter shaft through intracranial or extracranial neurovascular anatomy;
wherein the working lumen extends through the imaging region such that the catheter shaft is configured for over-the-wire advancement through the neurovascular guide catheter while the intravascular ultrasound imaging assembly is positioned for intraluminal ultrasound imaging; and
wherein the proximal hub assembly is configured to permit introduction of an acoustic coupling medium into at least one of the imaging lumen or the working lumen to support acquisition of the circumferential intraluminal ultrasound imaging data.

2. The intraluminal imaging delivery assist platform of claim 1, wherein the imaging lumen and the working lumen are arranged in a horizontal side-by-side configuration within the catheter shaft.

3. The intraluminal imaging delivery assist platform of claim 1, wherein the imaging lumen and the working lumen collectively define an oval-core dual-lumen arrangement within a circular outer diameter of the catheter shaft.

4. The intraluminal imaging delivery assist platform of claim 1, wherein the imaging lumen and the working lumen are separated by an extrusion boundary.

5. The intraluminal imaging delivery assist platform of claim 4, wherein the extrusion boundary comprises at least one of a polymer extrusion septum, a metallic hypotube septum, or a discrete shaped spine element disposed between the imaging lumen and the working lumen.

6. The intraluminal imaging delivery assist platform of claim 1, wherein the working lumen extends through the catheter shaft, the imaging region, and the distal tip region.

7. The intraluminal imaging delivery assist platform of claim 1, wherein the distal tip region comprises a working lumen centration transition configured to reposition the working lumen from a laterally offset position within the imaging region toward a centered position at a distal terminus of the distal tip region.

8. The intraluminal imaging delivery assist platform of claim 1, wherein the distal tip region comprises a distal taper extending from the imaging region toward a distal terminus of the distal tip region.

9. The intraluminal imaging delivery assist platform of claim 1, wherein the imaging lumen terminates proximal to a distal terminus of the distal tip region, and wherein the working lumen continues distally beyond the imaging lumen.

10. The intraluminal imaging delivery assist platform of claim 1, wherein the intravascular ultrasound imaging assembly comprises an IVUS drive cable and an ultrasound transducer disposed within the imaging lumen.

11. The intraluminal imaging delivery assist platform of claim 10, wherein the ultrasound transducer comprises a rotating piezoelectric transducer.

12. The intraluminal imaging delivery assist platform of claim 1, wherein the proximal hub assembly is configured to sequentially receive the microwire through the working port during advancement of the catheter shaft and receive the acoustic coupling medium through the working port after withdrawal of the microwire from the working lumen.

13. The intraluminal imaging delivery assist platform of claim 1, wherein the imaging lumen is configured to receive the acoustic coupling medium around the intravascular ultrasound imaging assembly, and wherein the working lumen is configured to receive the acoustic coupling medium after withdrawal of the microwire from the working lumen.

14. The intraluminal imaging delivery assist platform of claim 1, wherein the catheter shaft comprises a continuous outer polymer jacket extending along the imaging region and the distal tip region.

15. The intraluminal imaging delivery assist platform of claim 14, wherein the continuous outer polymer jacket comprises an acoustically transmissive material at the imaging region.

16. The intraluminal imaging delivery assist platform of claim 1, further comprising at least one radiopaque marker disposed adjacent to the imaging region or the distal tip region.

17. The intraluminal imaging delivery assist platform of claim 1, wherein the proximal hub assembly comprises a hub body, a strain relief coupled between the hub body and the catheter shaft, and a webbing zone extending between the working port and the imaging port.

18. The intraluminal imaging delivery assist platform of claim 17, wherein the proximal hub assembly comprises a hub body septum separating a working lumen path extending toward the working port from an imaging lumen path extending toward the imaging port.

19. An intraluminal imaging delivery assist catheter for neurovascular use, comprising:
a catheter body shaft configured for advancement through a neurovascular guide catheter, the catheter body shaft including an imaging lumen wall defining an imaging lumen and a working lumen wall defining a working lumen;
an extrusion boundary disposed between the imaging lumen wall and the working lumen wall, the imaging lumen wall and the working lumen wall being arranged side-by-side within a continuous outer polymer jacket to define an oval-core dual-lumen arrangement;
a proximal hub assembly coupled to the catheter body shaft, the proximal hub assembly including a working port in communication with the working lumen and an imaging port in communication with the imaging lumen;
an intravascular ultrasound drive cable disposed at least partially within the imaging lumen;
an intravascular ultrasound imaging assembly disposed at least partially within the imaging lumen and coupled to the intravascular ultrasound drive cable;
an imaging zone configured to acquire substantially circumferential intraluminal ultrasound imaging data from within neurovascular anatomy; and
a distal tip zone disposed distal to the imaging zone;
wherein the working lumen extends through the catheter body shaft, the imaging zone, and the distal tip zone to receive a microwire during over-the-wire advancement of the intraluminal imaging delivery assist catheter;
wherein the imaging lumen terminates proximal to a distal terminus of the distal tip zone; and
wherein the working lumen transitions within the distal tip zone from a laterally offset position relative to the imaging lumen toward a centered position at the distal terminus of the distal tip zone.

20. A method of performing intraluminal imaging using an intraluminal imaging delivery assist catheter, the method comprising:
advancing a neurovascular guide catheter through vasculature of a patient toward a target neurovascular region;
advancing the intraluminal imaging delivery assist catheter through the neurovascular guide catheter over a microwire, the intraluminal imaging delivery assist catheter including an imaging lumen, a working lumen arranged side-by-side with the imaging lumen, an intravascular ultrasound imaging assembly disposed at least partially within the imaging lumen, and a distal tip region;
positioning an imaging region of the intraluminal imaging delivery assist catheter within or adjacent to the target neurovascular region;
withdrawing the microwire from the working lumen after positioning the imaging region within or adjacent to the target neurovascular region;
introducing an acoustic coupling medium into the working lumen after withdrawing the microwire from the working lumen;
acquiring, using the intravascular ultrasound imaging assembly, substantially circumferential intraluminal ultrasound imaging data from the target neurovascular region; and
using the substantially circumferential intraluminal ultrasound imaging data to assess at least one of vessel morphology, vessel pathology, stenosis, thrombus, dissection, aneurysm neck configuration, vessel diameter, or device apposition.
